(19) United States
(12) Patent Application Publication
(10) Pub. No.: US 2022/0409319 A1
(43) Pub. Date: Dec. 29, 2022

(54) COLONOSCOPY AUXILIARY DEVICE

(71) Applicants: Brian Sehoon LIM, Irvine, CA (US); Micro-Tech (Nanjing) Co., Ltd., Nanjing (CN)

(72) Inventors: Brian Sehoon LIM, Irvine, CA (US); Scott HAACK, Chardon, OH (US)

(73) Assignees: Brian Sehoon LIM, Irvine, CA (US); Micro-Tech (Nanjing) Co., Ltd., Nanjing (CN)

(21) Appl. No.: 17/355,620

(22) Filed: Jun. 23, 2021

(51) Int. Cl. A61B 46/20; A61B 46/00

(52) U.S. Cl. CPC A61B 46/20; A61B 46/40; A61B 2046/205; A61B 1/31

(57) ABSTRACT

A colonoscopy auxiliary device. The colonoscopy auxiliary device includes: a foldable shield portion provided with a colo-examination aperture; and a first adhesive strip disposed on a first edge of the foldable shield portion.

### TECHNICAL FIELD

Embodiments of the present disclosure relate to the field of colonoscopy, and in particular, to a colonoscopy auxiliary device.

### BACKGROUND

Colonoscopy is a commonly used fiber endoscope. Retrograde insertion of the colonoscopy through the anus can check the rectum, sigmoid colon, descending colon, transverse colon, ascending colon, and cecum, and a small intestine connected to the large intestine. Intestinal lesions can be clearly found by the colonoscopy, and some intestinal lesion can be treated at the same time. For example, benign lesions such as large intestine polyps are resected, intestinal bleeding is treated with hemostasis, and foreign bodies in the large intestine are removed using various accessories.

Breathing, coughing and secretions of patients with COVID-19 cause viral aerosols and contaminants to float in the air and soil the working space, causing pollution to the surrounding environment. In the clinical work of the current epidemic situation, it is particularly important to do a good job in response and protection of physicians and staff.

### SUMMARY

Embodiments of the present application provide a colonoscopy auxiliary device.

In a first aspect, a colonoscopy auxiliary device is provided, the colonoscopy auxiliary device includes: a foldable shield portion provided with a colo-examination aperture; and a first adhesive strip disposed on a first edge of the foldable shield portion.

In an implementation, the colonoscopy auxiliary device further includes a second adhesive strip disposed on a second edge of the foldable shield portion, where the second adhesive strip intersects with the first adhesive strip, or an extension line of the second adhesive strip intersects with an extension line of the first adhesive strip.

In an implementation, the foldable shield portion is a quadrilateral sheet, the first adhesive strip and the second adhesive strip are disposed on the first edge and the second edge of the foldable shield portion that are adjacent to each other, respectively, where the quadrilateral sheet is a rectangle sheet or a square sheet.

In an implementation, the colonoscopy auxiliary device further includes at least one auxiliary adhesive strip, where the at least one auxiliary adhesive strip includes at least one of a first auxiliary adhesive strip and a second auxiliary adhesive strip; where, the first auxiliary adhesive strip is disposed on the first edge, where the first adhesive strip and the first auxiliary adhesive strip are disposed in parallel, and a first perforation line is disposed on the foldable shield portion and located between the first adhesive strip and the first auxiliary adhesive strip; the second auxiliary adhesive strip is disposed on the second edge, where the second adhesive strip and the second auxiliary adhesive strip are disposed in parallel, and a second perforation line is disposed on the foldable shield portion and located between the second adhesive strip and the second auxiliary adhesive strip.

In an implementation, an area surrounding the colo-examination aperture is thicker than other portion of the foldable shield portion; where the area surrounding the colo-examination aperture has a multiple-layer structure, or has a single layer with thicker polymer.

In an implementation, the colonoscopy auxiliary device further includes a flap, where the flap is configured to cover the colo-examination aperture.

In an implementation, the colonoscopy auxiliary device further includes a handle element, where the flap is disposed on the foldable shield portion, and the handle element is disposed on an upper surface of the flap for moving the flap to cover or expose the colo-examination aperture.

In an implementation, a first tape is disposed around the colo-examination aperture and configured to fix the flap over the colo-examination aperture to cover the colo-examination aperture; or the flap is fixed rotatably on the foldable shield portion to cover and expose the colo-examination aperture alternatively.

In an implementation, a third perforation line is disposed on the foldable shield portion from an edge of the colo-examination aperture to an edge of the foldable shield portion opposite to the first edge.

In an implementation, the colonoscopy auxiliary device further includes a second tape, where the second tape is disposed along the third perforation line and configured to reattach the third perforation line after the third perforation line is ripped.

In an implementation, the colonoscopy auxiliary device further includes a chuck portion, where the chuck portion is connected to an edge of the foldable shield portion opposite to the first edge.

In an implementation, a fourth perforation line is disposed on a connection portion of the chuck portion and the foldable shield portion to separate the chuck portion and the foldable shield portion.

In an implementation, the chuck portion includes a back sheet and a top sheet, and the top sheet is attached on the back sheet; where the back sheet is made of polypropylene, and the top sheet is made of hydrophilic non-woven.

In an implementation, the colonoscopy auxiliary device further includes a string, disposed around a perimeter of the foldable shield portion and the chuck portion to string the foldable shield portion and the chuck portion together, and exposed from positions on a second edge of the foldable shield portion and an edge opposite to the second edge to facilitate the string being pulled, where the positions correspond to the fourth perforation line.

In an implementation, two third tapes are disposed on a second edge and an edge opposite to the second edge, respectively.

In an implementation, at least one fourth tape is disposed on an edge of the foldable shield portion opposite to the first edge.

In an implementation, the foldable shield portion includes: an absorbent layer and a water barrier layer, where the first adhesive strip is disposed on the absorbent layer; or an absorbent layer, a water barrier layer and a non-woven layer, where the water barrier layer is disposed between the absorbent layer and the non-woven layer, and the first adhesive strip is disposed on the absorbent layer.

In an implementation, the foldable shield portion includes a colo-examination area, where the colo-examination aperture is disposed in the colo-examination area, and at least the colo-examination area of the foldable shield portion is transparent.

In an implementation, the colonoscopy auxiliary device further includes a third adhesive strip disposed on an edge of the foldable shield portion opposites to the second edge.

In an implementation, the colonoscopy auxiliary device further includes at least one of a third adhesive strip and a fourth adhesive strip, where: the third adhesive strip is disposed on an edge of the foldable shield portion opposite to the second edge, and the device further includes a continuous non-stick plastic film, where the continuous non-stick plastic film is configured to cover the first adhesive strip, the second adhesive strip, the third adhesive strip; and the fourth adhesive strip is disposed on an edge of the foldable shield portion opposite to the first edge, and the device further includes a continuous non-stick plastic film, where the continuous non-stick plastic film is configured to cover the first adhesive strip, the second adhesive strip, the third adhesive strip and the fourth adhesive strip.

In an implementation, at least one corner end of the continuous non-stick plastic film is formed with a protrusion, where the at least one corner end of the continuous non-stick plastic film corresponds to at least one of: an intersection point of the first adhesive strip and the second adhesive strip, an intersection point of the first adhesive strip and the third adhesive strip, an intersection point of the fourth adhesive strip and the second adhesive strip, and an intersection point of the fourth adhesive strip and the third adhesive strip.

In an implementation, at least one corner end of the continuous non-stick plastic film is formed with a non-stick plastic crease; where the at least one corner end of the continuous non-stick plastic film corresponds to at least one of: an intersection point of the first adhesive strip and the second adhesive strip, an intersection point of the first adhesive strip and the third adhesive strip, an intersection point of the fourth adhesive strip and the second adhesive strip, and an intersection point of the fourth adhesive strip and the third adhesive strip.

### BRIEF DESCRIPTION OF THE DRAWINGS

In order to illustrate the embodiments of the present application or the technical solutions in the prior art clearer, the accompanying drawings used in the description of the embodiments or the prior art will be briefly illustrated below. Obviously, the accompanying drawings in the following description are some embodiments of the present application, and other accompanying drawings can be obtained by those skilled in the art according to the accompanying drawings without any creative work.

FIG. 1 is a schematic structural diagram of a colonoscopy auxiliary device according to an embodiment of the present disclosure;

FIG. 2 is a schematic structural diagram of a colonoscopy auxiliary device according to an embodiment of the present disclosure;

FIG. 3 is a schematic structural diagram of a colonoscopy auxiliary device according to another embodiment of the present disclosure;

FIG. 4(a) is a schematic structural diagram of a colo-examination aperture with a flap covering thereon according to an embodiment of the present disclosure;

FIG. 4(b) is a schematic structural diagram of a colo-examination aperture with a flap covering thereon according to an embodiment of the present disclosure

FIG. 5 is a schematic structural diagram of a colonoscopy auxiliary device with a string according to an embodiment of the present disclosure;

FIG. 6 is a schematic structural diagram of a colonoscopy auxiliary device according to an embodiment of the present disclosure;

FIG. 7 is a schematic structural diagram of a colonoscopy auxiliary device with continuous non-stick plastic film according to embodiment of the present disclosure;

FIG. 8 is a schematic structural diagram of a colonoscopy auxiliary device with continuous non-stick plastic film according to another embodiment of the present disclosure;

FIG. 9 is a schematic structural diagram of fourth tape according to another embodiment of the present disclosure.

### DETAILED DESCRIPTION OF THE EMBODIMENTS

In order to make the purpose, technical solutions and advantages of the embodiments of the present disclosure clearer, the technical solutions of the embodiments of the present disclosure will be clearly and completely described below with reference to the accompanying drawings in the embodiments of the present disclosure. Apparently, the described embodiments are part of the embodiments of the present application, rather than all of the embodiments. On the basis of the embodiments of the present application, all other embodiments obtained by those skilled in the art without creative work fall within the protection scope of the present application.

In the present application, “at least one” means one or more, and “a plurality of” means two or more. “And/or” describes the association relationship of the associated objects, which indicates that there can be three relationships. For example, A and/or B can indicate that A exists separately, A and B exist at the same time, and B exists separately, where A and B can be singular or plural. The character “/” generally indicates that the contextual objects are an “or” relationship. “At least one of the following” or a similar expression thereof refers to any combination of these items, including any combination of a single item or a plurality of items. For example, at least one of a, b, or c can represents: a, b, c, a and b, a and c, b and c, or a, b and c, where a, b, and c may be single or multiple. In the present application, the terms “first”, “second”, and the like, are used to distinguish similar objects, and are not necessarily used to describe a particular order or a sequence.

Currently, COVID-19 has changed the world including the practice of gastroenterology. There have been numerous articles that talked about the gastrointestinal symptoms of COVID including diarrhea, nausea and/or vomiting, anorexia. There is a concern for GI procedures including EGD and colonoscopy.

Below are some articles that have been published:

Repici et al. Coronavirus (COVID-19) outbreak: what the department of endoscopy should know. Gastrointest Endoscopy. 2020 Mar. 13. 1) Endoscopy should be considered high risk procedure; 2) Endoscopy requires short physical distance from patients to the personnel. According to studies performed during global SARS 2003, droplets from infected patients could reach persons located 6 feet or more from the source; 3) Possible fecal-oral transmission; 4) Transmission can occur during incubation period in asymptomatic patients.

COVID-19: Gastrointestinal manifestations and potential fecal-oral transmission. (Gu et al) Gastroenterology. 2020 Mar. 3. 1) The first case of 2019-nCoV infection confirmed in the United States reported a 2-day history of nausea and vomiting on admission, and then passed a loose bowel movement on hospital day 2. The viral nucleic acids of loose stool and both respiratory specimens later tested positive; 2) 2019-nCoV sequence could be also detected in the self-collected saliva of most infected patients even not in nasopharyngeal aspirate; 3) Digestive system other than respiratory system may serve as an alternative route of infection when people are in contact with infected animals and/or people—asymptomatic carriers or individuals with mild enteric symptoms at an early stage may have been neglected or underestimated in previous investigations; 4) To date, molecular model have revealed by the next-generation sequencing technology that 2019-nCoV shares about 79% sequence identify to SARS-CoV indicative of these 2 lineage B β-coronaviruses highly homologous-angiotensin converting enzyme II (ACE2) previously known as an entry receptor for SARS-CoV was exclusively confirmed in 2019-nCoV infection despite amino acid mutations at some key receptor-binding domains; 5) Recent bioinformatics analysis on available single-cell transcriptomes data of normal human lung and gastrointestinal system was carried out to identify the ACE2-expressing cell composition and proportion, and revealed that ACE2 was not only highly expressed in the lung AT2 cells, but also in esophagus upper and stratified epithelial cells and absorptive enterocytes from ileum and colon.

Effect of gastrointestinal symptoms on patients infected with COVID-19 (Zhou et al). Gastroenterology. 2020 Mar. 18. RT-PCR positive findings from patients' stool revealed that COVID-19 may spread by fecal-oral transmission.

Considerations in performing endoscopy during the COVID-19 pandemic. (Soetikno, et al. 2020 Mar. 27. 1) Transmission can occur in both symptomatic and asymptomatic individuals; 2) Viable virus particles can be detected in aerosols up to 3 hours after aerosolization and up to 3 days on surfaces; 3) The risk of infection to healthcare workers is significant: in one of the earliest documentations of infection in Wuhan, 29% of patients (40 out of 138) were healthcare workers; 4) It is unknown how much of the risk was related to the direct care of infected patients or to the inadequate use of personal protective equipment (PPE); 5) All endoscopic procedures should be considered aerosol-generating procedures (AGP), a) Coughing and retching can occur during upper endoscopy, generating aerosols, b) Patients undergoing colonoscopy may pass flatus, which is also known to disseminate bacteria to nearby surroundings; 6) World Health Organization (WHO) has published an extensive guideline on the rational use of personal protective equipment (PPE) for COVID-19 and provided specific instructions for healthcare workers performing AGP on patients with COVID-19, Use of a respirator (N95, FFP2 standard, or equivalent), gown, gloves, eye protection, and apron although aprons are not usually not used in the United States; 7) Surface contamination during endoscopy: stool mixed with water often drips to the bed during colonoscopy; 8) Contamination of the room used by patients with COVID-19: Extensive environmental contamination can occur even from patients with mild COVID-19 upper respiratory symptoms. Ong and colleagues 15 detected positive SARS-CoV-2 samples in various locations around the patient's room, including the patient's bed, sink, bathroom, light switches, and doors. In addition, positive samples were found on the shoes and stethoscope of staff exiting the patient's room. However, there was no contamination in the anteroom or corridor outside the room. The study illustrates the significant extent of contamination by patients with SARS-CoV-2 through respiratory droplets and fecal shedding.

Based on the above, there is some need to be improved: a) For EGD, many physicians have been using face masks instead of nasal cannulas. This can potentially decrease physician or staff exposure (especially when patients are coughing, gagging). However, there is no “mask” that we can put on patients for colonoscopy; b) During colonoscopy, create a barrier between patients' buttocks and physician given that physician usually stands within a foot from the patient; c) By creating this barrier, physician gets less exposure to stool and flatus; d) Design has to be simple enough but able to provide adequate protection; e) Design has to be easily disposable.

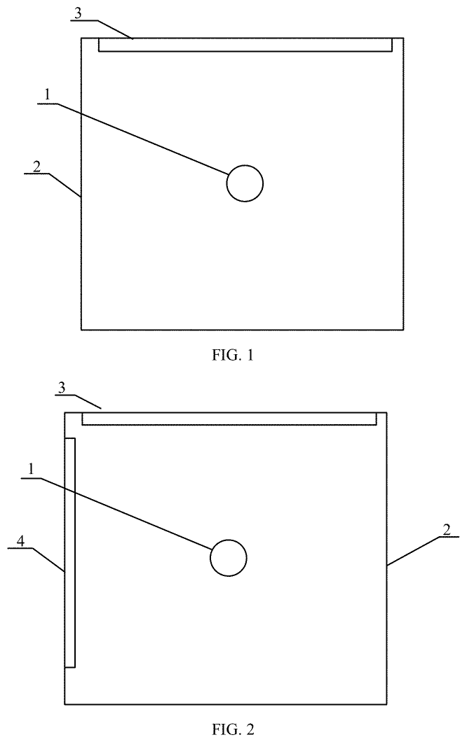

Embodiments of the present disclosure provide a colonoscopy auxiliary device. FIG. 1 is a schematic structural diagram of a colonoscopy auxiliary device according to an embodiment of the present disclosure. As illustrated in FIG. 1, the colonoscopy auxiliary device can include: a foldable shield portion and a first adhesive strip 3, where the foldable shield portion 2 is provided with a colo-examination aperture 1, and the first adhesive strip 3 is disposed on a first edge of the foldable shield portion 2.

In the colonoscopy auxiliary device of the embodiment shown in FIG. 1, the foldable shield portion 2 is provided with the colo-examination aperture 1. During an operation, the foldable shield portion 2 is placed above a patient's body (for example, above or around the patient's buttocks) and the first adhesive strip 3 is posted on the patient, so that a colonoscope can enter the patient's body through the colo-examination aperture 1 without exposing the patient's body directly to an endoscopist, thereby significantly decreasing the endoscopist's exposure to stool and flatus. The first adhesive strip 3 can be posted along a proper portion of the patient, for example, the first adhesive strip 3 is posted on the patient's thigh to fix the foldable shield portion 2. In other implementation, the first adhesive strip 3 is posted on the other proper portion of the patient to fix the foldable shield portion 2, as long as the colonoscope can enter the patient's body through the colo-examination aperture 1 without exposing the patient's body directly to an endoscopist.

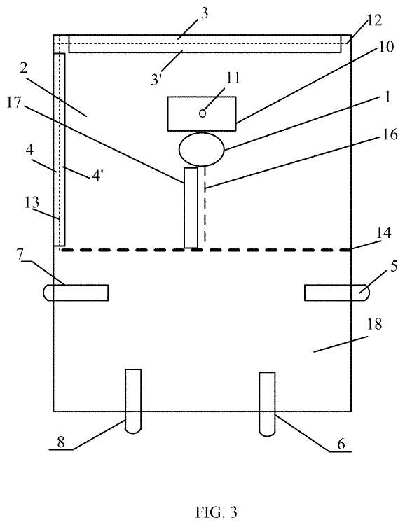

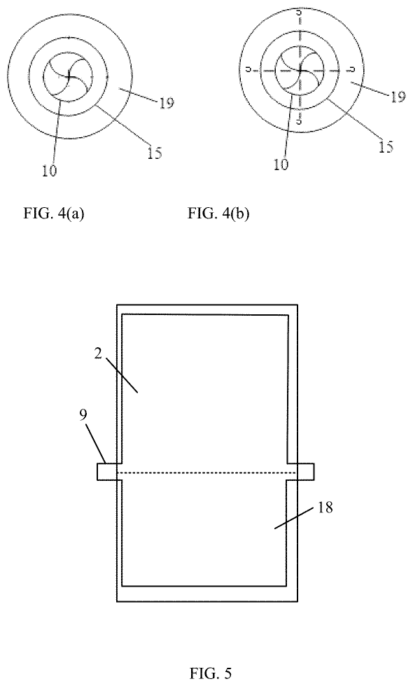

FIG. 2 is a schematic structural diagram of a colonoscopy auxiliary device according to another embodiment of the present disclosure. FIG. 3 is a schematic structural diagram of a colonoscopy auxiliary device according to another embodiment of the present disclosure, FIGS. 4(a) and 4(b) are schematic structural diagrams of a colo-examination aperture with a flap covering thereon according to an embodiment of the present disclosure, and FIG. 5 is a schematic structural diagram of a colonoscopy auxiliary device with a string according to an embodiment of the present disclosure. As illustrated in FIG. 2, the colonoscopy auxiliary device of this embodiment can further include a second adhesive strip 4 disposed on a second edge of the foldable shield portion 2, and an extension line of the second adhesive strip 4 intersects an extension line of the first adhesive strip 3. The direction of the extension line of the first adhesive strip 3 is the direction of a line where the first adhesive strip 3 is located, and the direction of the extension line of the second adhesive strip 4 is the direction of a line where the second adhesive strip 4 is located. During the operation, the first adhesive strip 3 is posted along the patient's thigh, while the second adhesive strip 4 is posted on the patient's back, so that the foldable shield portion 2 could enclose the patient's body in a more stable way, thereby further decreasing the endoscopist's exposure to stool and flatus.

In an implementation, the foldable shield portion 2 may be a quadrilateral sheet, such as a rectangle sheet or a square sheet, and the first adhesive strip 3 and the second adhesive strip 4 are disposed on the first edge and the second edge of the foldable shield portion that are adjacent to each other, respectively, as shown in FIGS. 2 and 3.

In an implementation, the foldable shield portion 2 is made of thin plastic which is easily collapsible at the end of the operation. In an implementation, the foldable shield portion 2 may be made of polymer.

In an implementation, the foldable shield portion 2 includes an absorbent layer and a water barrier layer, which are stacked with each other. The absorbent layer is smaller than the water barrier layer, and the adhesive strips are disposed on a side of the water barrier layer on which the absorbent layer is stacked. In an example, the adhesive strips are disposed on edges of the water barrier layer and surround the water barrier. The absorbent layer comes into contact with the patient during the operation. In another implementation, the foldable shield portion 2 further includes a non-woven layer, which is stacked on the water barrier layer. In this case, the water barrier layer is disposed between the absorbent layer and the non-woven layer.

In an implementation, the foldable shield portion can include a colo-examination area 19. The colo-examination aperture 1 is disposed in the colo-examination area 19, and in an implementation, at least the colo-examination area 19 of the foldable shield portion 2 can be a transparent area to facilitate the operation. In an example, the entire foldable shield portion 2 is made of transparent material. In another example, the colo-examination area 19 is made of transparent material and the other area of the foldable shield portion 2 is made of non-transparent material.

In an implementation, as illustrated in FIGS. 2 and 3, the foldable shield portion 2 is rectangle, the first adhesive strip 3 is disposed on a long edge of the rectangle, and the second adhesive strip 4 is disposed on a short edge of the rectangle. In an implementation, the long edge may be 600 mm, and the short edge may be 400 mm, and the first adhesive strip 3 may be 300 mm in length. In an implementation, the second adhesive strip 4 has the same length with the short edge. In an implementation, the diameter of the colo-examination aperture 1 may be 9.5 mm.

At least one auxiliary adhesive strip is disposed on the first edge and/or the second edge. In an implementation, a first auxiliary adhesive strip 3′ is further disposed on the first edge of the foldable shield portion 2 and a second auxiliary adhesive strip 4′ is further disposed on the second edge of the foldable shield portion 2. The first adhesive strip 3 and the first auxiliary adhesive strip 3′ are disposed in parallel and the second adhesive strip 4 and the second auxiliary adhesive strip 4′ are disposed in parallel; and a first perforation line 12 is disposed between the first adhesive strip 3 and the first auxiliary adhesive strip 3′, and a second perforation line 13 is disposed between the second adhesive strip 4 and the second auxiliary adhesive strip 4′. In another implementation, the at least one auxiliary adhesive strip can only include the first auxiliary adhesive strip 3′, which is disposed on the first edge of the foldable shield portion 2, but is not limited herein.

As can be seen in FIG. 3, the first adhesive strip 3 and the first auxiliary adhesive strip 3′ are disposed on the first side of the foldable shield portion 2 in parallel, and the first perforation line 12 is disposed between the first adhesive strip 3 and the first auxiliary adhesive strip 3′ and extends from the second edge of the foldable shield portion 2 to the edge opposite to the second edge; the second adhesive strip 4 and the second auxiliary adhesive strip 4′ are disposed on the second side of the foldable shield portion 2 in parallel, and the second perforation line 13 is disposed between the second adhesive strip 4 and the second auxiliary adhesive strip 4′ and extends from the first edge to the edge of the foldable shield portion 2 opposite to the first edge.

During the operation, at first, the second adhesive strip 4 is posted draping down on the patient's back, and the first adhesive strip 3 is posted along the patient's thigh. The design of the first auxiliary adhesive strip 3′ and the second auxiliary adhesive strip 4′ contributes to the colonoscopy auxiliary device that can be used twice if the patient has to be turned, etc during the operation. For example, in the situation that the patient changes posture from lie on one side to other side, and the first adhesive strip 3 and/or the second adhesive strip 4 do not stick anymore and should be detached, the first adhesive strip 3 can be detached off by the first perforation line 12, and the second adhesive strip 4 can be detached off by the second perforation line 13. At this time, the first auxiliary adhesive strip 3′ and the second auxiliary adhesive strip 4′ can play a role and replace the first adhesive strip 3 and the second adhesive strip 4 for use, for example, the first auxiliary adhesive strip 3′ can now be posted draping down on the patient's back, and the second auxiliary adhesive strip 4′ can now be posted along the patient's thigh. In this way, the colonoscopy auxiliary device can be used twice during the operation, thereby saving the costs while improving the hygiene.

It should be understood that there may be more first auxiliary adhesive strip 3′ and more second auxiliary adhesive strip 4′ disposed on the foldable shield portion 2, which is not limited in the embodiments of the present disclosure.

In an implementation, the area surrounding the colo-examination aperture 1 is thicker than other portion of the foldable shield portion 2. For example, the area surrounding the colo-examination aperture 1 is a reinforced built-up area with a thicker polymer where the colonoscopy is placed; for another example, the area surrounding the colo-examination aperture has a multiple-layer structure, so that forces from the colonoscopy during the operation would not rip the edge of colo-examination aperture 1 unnecessarily.

In an implementation, the reinforced colo-examination aperture 1 is disposed in the middle of the foldable shield portion 2. During the operation, the foldable shield portion 2 is placed with the reinforced colo-examination aperture 1 corresponding to the patient's anus, while the endoscopist's finger or the colonoscopy can get through the reinforced colo-examination aperture 1 and then enter the patient via anus.

According to some implementations, the foldable shield portion may further include a flap 10, which is configured to cover or expose the colo-examination aperture 1. The flap 10 may be in shape of square or round, and the size of the flap 10 is larger than the size of the colo-examination aperture 1 so that the flap 10 can cover the colo-examination aperture 1 entirely. The shape of the flap 10 is not limited in the embodiments of the present disclosure as long as the flap 10 can cover the colo-examination aperture 1.

In an implementation, as illustrated in FIGS. 3 and 4, the flap 10 is disposed near the colo-examination aperture 1 and configured to close the colo-examination aperture 1. In an implementation, the flap 10 is fixed rotatably on the foldable shield portion 2 so as to cover and expose the colo-examination aperture 1 alternatively. For example, the flap 10 is fixed over the colo-examination aperture 1 by a fix element (not shown), and the flap 10 can rotate around the fix element to open and close the colo-examination aperture 1 if needed. For example, the flap 10 is fixed rotatably to open the colo-examination aperture 1 at the beginning of the operation, and close the colo-examination aperture 1 at the end of the operation. With the above method, the device can collapse away from the patient to not soil the endoscopist. In other embodiments, the flap 10 may be secured on the foldable shield portion 2 through other methods, which is not limited.

In an implementation, a first tape 15 is disposed around the colo-examination aperture 1 and configured to fix the flap 10 over the colo-examination aperture 1 to cover the colo-examination aperture 1. For example, as illustrated in FIG. 4 (a), the flap 10 is round, and the first tape 15 which has non-stick plastic film thereon is disposed around the colo-examination aperture 1 (illustrated in FIG. 1). At the end of the operation, the non-stick plastic film can be peeled of, and the flap 10 can be secured onto the foldable shield portion 2 by the first tape 15.

In an implementation, a handle element is disposed on an upper surface of the flap 10 for moving the flap 10 to cover the colo-examination aperture 1. As illustrated in FIG. 3, a ring 11 is disposed on the upper surface of the flap 10, with which the fingers of the endoscopist can move the flap 10 to open and close the colo-examination aperture 1. In other embodiments, other elements may be disposed on the upper surface of the flap 10 to facilitate the grabbing by the fingers, and these elements are not limited to the ring. In an implementation, the handle element allows the endoscopist to close the colo-examination aperture 1 after the colonoscopy is pulled out of the colo-examination aperture 1, so that the foldable shield portion 2 can collapse away from the patient to not soil the endoscopist.

In an implementation, the colo-examination area 19 is configured as a transparent silicone pad, and the colo-examination aperture 1 is an aperture cross. As illustrated in FIG. 4(b), each of the tail of the aperture cross is round to prevent tearing. In this case, the flap 10 and the first tape 15 may exist or not exist.

According to some implementations, the reinforced colo-examination aperture 1 is disposed in the middle of the foldable shield portion 2, and the colonoscopy can get through the reinforced colo-examination aperture 1 and then enter the patient via his/her anus. During the operation, when there is a situation in which the device might have to be removed and repositioned from the patient, for example, in case of a patient movement and the like in which the patient's position or posture may change, the foldable shield portion 2 may be removed and repositioned from the patient. In this case, the foldable shield portion 2 can be separated on either side of the colonoscope. In an implementation, the foldable shield portion 2 may further include a third perforation line 16, which is disposed on the foldable shield portion 2 from an edge of the colo-examination aperture 1 to an edge of the foldable shield portion 2 opposite to the first edge. For example, if the patient has to move, the third perforation line 16 can be ripped staring from the edge of the colo-examination aperture 1, so that the colonoscope can be freed.

In an implementation, the foldable shield portion 2 may further include a second tape 17, which is disposed along the third perforation line 16 and configured to reattach the third perforation line 16 after the third perforation line 16 is ripped. For example, as illustrated in FIG. 3, the second tape 17 is disposed along the third perforation line 16 with non-stick plastic film thereon, so that the foldable shield portion 2 can be re-joined around the colonoscope once the patient's position has been adjusted. For example, at the point after the patient's position is adjusted, the non-stick plastic film can be peeled off and the second tape can be exposed to reattach the third perforation line 16 and secure the foldable shield portion 2 properly around the colonoscope once again, thereby preventing from having to remove the colonoscope and apply a new colonoscope auxiliary device.

In some embodiments, more than one of the second tapes 17 are disposed along the third perforation line 16 and configured to reattach the third perforation line 16 if the third perforation line 16 need to be ripped for more than once in the case that the patient needs to move for more times, the physician can use second tapes 17 to reattach the third perforation line 16 together.

It should be understood that in some embodiments, it would not be allowed for the same foldable shield portion 2 to be used twice or more times on the same patient, in this situation, more extra second tapes may be used on the foldable shield portion 2, and if the foldable shield portion 2 has to be separated again at the third perforation line 16 the second or third time, the extra second tapes can be used to reattach the third perforation line 16 together. But if the patient has to be re-positioned, a new foldable shield portion 2 has to be applied again, and in this case the third perforation line 16 may not be provided.

According to some implementations, the colonoscopy auxiliary device may further include a chuck portion 18, which is connected to an edge of the foldable shield portion 2 opposite to the first edge. In an implementation, the chuck portion 18 may also be a quadrilateral sheet, such as a rectangle sheet or a square sheet. As illustrated in FIG. 3, for example, the chuck portion 18 is connected or attached to the foldable shield portion 2 at the edge of the foldable shield portion 2 opposite to the first edge; the second edge and the edge of the foldable shield portion 2 opposite to the second edge extend to form two edges of the chuck portion 18. That is, the second edge of the foldable shield portion 2 and the second edge of the chuck portion 18 form the second edge of the device, the first edge of the foldable shield portion 2 forms the first edge of the device, the edge of the foldable shield portion 2 opposite to the second edge of the foldable shield portion 2 and the edge of the chuck portion 18 opposite to the second edge of the chuck portion 18 form the edge of the device opposite to the second edge of the device.

In an implementation, a fourth perforation line 14 is disposed in a connection portion of the chuck portion 18 and the foldable shield portion 2 so as to separate the chuck portion 18 and the foldable shield portion 2 when needed. As illustrated in FIG. 3, for example, the chuck portion 18 is connected or attached to the foldable shield portion 2 at the connection portion with the fourth perforation line 14, and the fourth perforation line 14 extends from the second edge of the foldable shield portion 2 to the edge opposite to the second edge. The fourth perforation line 14 is considered a way to separate the chuck portion 18 from the foldable shield portion 2 so that the colonoscopy can be completely freed if the patient has to be moved.

In an implementation, a velcro is optioned to attach the device to the bed sheet. For example, the velcro attaches the device to the bed. In another implementation, a plastic clip may be disposed on an edge of the device to clip the device onto a bed sheet, which can further effectively avoid the device from coming off too easily. In other embodiments, other elements may be adopted to replace the plastic clip so that the device can be clipped on to the bed sheet or chucks, which is not limited.

In an implementation, the chuck portion 18 includes a back sheet and a top sheet, and the top sheet is attached on the back sheet. In an implementation, the back sheet is made of polypropylene, and the top sheet is made of hydrophilic non-woven.

During the operation, a portion of the chuck portion 18 is able to be inserted underneath the patient. Compared with the prior art, in which usually the endoscopist uses a towel with chucks (like a fluid absorbing pad that the patient lays on) over it to absorb bodily fluids so that the bodily fluids can be easily disposed of at the end of the operation, the hydrophilic non-woven laying under the patient in this embodiment of the present disclosure would be applied to the buttock or the backside of the patient to create a larger barrier to envelope the biomatter expelled by the patient.

According to some implementations, the colonoscopy auxiliary device may further include a string 9, which is disposed around a perimeter of the foldable shield portion 2 and the chuck portion 18 to string the foldable shield portion 2 and the chuck portion 18 together. The string 9 is configured to be exposed from positions on the second edge of the foldable shield portion and the edge opposite to the second edge to facilitate the string being pulled. In an implementation, the positions correspond to the fourth perforation line 14, that is, the string 9 is configured to expose on positions of the second edge and the edge opposite to the second edge corresponding to the fourth perforation line 14. As illustrated in FIG. 5, the string 9 is disposed running along the perimeter of the entirety of the foldable shield portion 2 and the chuck portion 18 so that the colonoscopy auxiliary device can be collapsed at the conclusion of the operation. The string 9 is configured to detach the device from the patient when being pulled with fingers, meanwhile the excrement expelled by the patient during the operation can be collected together and properly disposed of by the device.

In some implementations, a “roll then tape” mechanism is provided. In an implementation, the two third tapes are disposed on the second edge of the device and the edge opposite to the second edge, respectively, for example, the two third tapes are disposed on positions on the second edge and the edge opposite to the second edge corresponding to the fourth perforation line 14. In an implementation, at least one fourth tape is disposed on an edge of the chuck portion away from the first edge.

As illustrated in FIG. 3, a third tape 7 is disposed on the second edge of the device, and a third tape 5 is disposed on the edge opposite to the second edge of the device. For example, the third tape 7 with non-stick plastic film thereon is disposed on a portion of the second edge distal from the first edge, and the third tape 5 with non-stick plastic film thereon is disposed on a portion of the edge that is opposite to the second edge and distal from the first edge, as shown in FIG. 3. In an implementation, the two third tapes 5 and 7 are disposed corresponding to the fourth perforation line 14.

At least one fourth tape is disposed on an edge of the foldable shield portion 2 opposite to the first edge. In the embodiment illustrated in FIG. 3, at least one fourth tape is disposed on an edge of the chuck portion away from the fourth perforation line 14. In an implementation, a fourth tape 6 and a fourth tape 8 are disposed on the edge of the chuck portion that is away from the first edge. For example, as illustrated in FIG. 3, the fourth tape 6 and the fourth tape 8 with non-stick plastic film thereon are disposed on the edge opposite to the first edge with the first adhesive strip 3.

At the conclusion of the operation, the device is rolled away from the endoscopist so that the physician contamination is minimized. When the device is rolled, the device can be closed off with the third tape 5 and the third tape 7. Once the device is rolled all the way down from the first edge to the part where the foldable shield portion 2 is connected to the chuck portion 18, the chuck portion 18 can be pulled from the space between the patient and the bed. Then the foldable shield portion 2 that has been rolled can continually be rolled together with the chuck portion 18, and the device can be closed off with the fourth tape 6 and the fourth tape 8.

The mechanism with the third and/or fourth tapes has the advantage of saving production cost, and potentially it can be easier to handle by the endoscopist. It should also be understood that the mechanism with the third and/or fourth tapes and the mechanism with the string 9 can be used separately or in combination.

A method for operating the colonoscopy auxiliary device according to an embodiment of the present disclosure is described as follows. During an operation, first the colonoscopy auxiliary device is unfolded on a bed. Then a patient lies on the chuck portion 18 with the hydrophilic non-woven so that the chuck portion is inserted underneath the patient. The first adhesive strip 3 is posted along the patient's thigh, and the second adhesive strip 3 is posted draping down on the patient's back. Before using the colonoscope, the flap 10 is in open position to expose (or open) the colo-examination aperture 1, and then the colonoscope gets into the patient's body through the colo-examination aperture 1 under the operation by the endoscopist. When the colonoscope is working, the flap 10 with the ring 11 keeps exposing the colo-examination aperture 1. If the patient has to move, the third perforation line 16 can be ripped staring from the colo-examination aperture 1, so that the colonoscope can be freed, and then the second tape 17 can help to reattach the third perforation line 16 and secure the foldable shield portion 2 properly around the colonoscope once again; or the fourth perforation line 14 is ripped to separate the chuck portion 18 and the foldable shield portion 2, so that the colonoscope can be completely freed.

When the operation is finished, the endoscopist pulls out the colonoscope from the colo-examination aperture 1 as the string 9 is being pulled, then moves the flap 10 via the ring 11 to cover the colo-examination aperture 1, and then removes the device from the body of the patient. At the end of the operation, the device is folded up to wrap the stool.

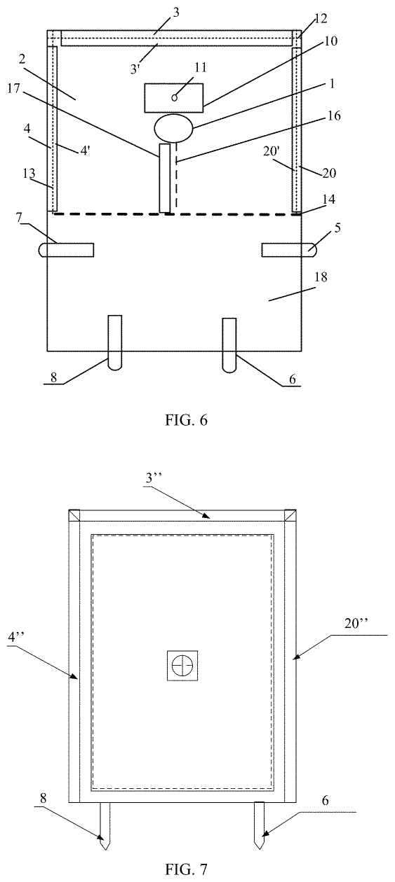

FIG. 6 is a schematic structural diagram of a colonoscopy auxiliary device according to another embodiment of the present disclosure. Based on the embodiments illustrated in FIGS. 1-5, the colonoscopy auxiliary device further includes a third adhesive strip 20. Reference may be made to the above device embodiments for the operations of the related device and the similar expressions. As illustrated in FIG. 2, the third adhesive strip 20 is disposed on an edge of the foldable shield portion 2 opposite to the second edge.

In an implementation, a third auxiliary adhesive strip 20′ is further disposed on edge of the foldable shield portion 2 that opposites to the second edge. The third adhesive strip 20 and the third auxiliary adhesive strip 20′ are disposed in parallel. In another implementation, no auxiliary adhesive strip is disposed on edge of the foldable shield portion 2 that opposites to the second edge, and only the third adhesive strip 20 is disposed, which is not limited herein.

During the operation, the third adhesive strip 20 and/or the third auxiliary adhesive strip 20′ are posted draping down on the patient's legs, so that the foldable shield portion 2 can further enclose the patient's body completely, thereby significantly decreasing the endoscopist's exposure to stool and flatus.

FIG. 7 is a schematic structural diagram of a colonoscopy auxiliary device with continuous non-stick plastic film according to an embodiment of the present disclosure. Based on any of the embodiments illustrated in FIGS. 1-6, the colonoscopy auxiliary device further includes continuous non-stick plastic films configured to cover the first adhesive strip, the second adhesive strip and the third adhesive strip. Reference may be made to the above device embodiments for the operations of the related device and the similar expressions. As illustrated in FIG. 7, for example, the continuous non-stick plastic films includes a first non-stick plastic film 3″ covering the first adhesive strip 3 and the first auxiliary adhesive strip 3′, a second non-stick plastic film 4″ covering the second adhesive strip 4 and the second auxiliary adhesive strip 4′, and a third non-stick plastic film 20″ covering the third adhesive strip 20 and the third auxiliary adhesive strip 20′, where the first non-stick plastic film 3″ is connected to the second non-stick plastic film 4″ at the first corner end, and the first non-stick plastic film 3″ is connected to the third non-stick plastic film 20″ at the second corner end.

At least one corner end of the continuous non-stick plastic film is formed with a protrusion, where the at least one corner end of the continuous non-stick plastic film corresponds to at least one of: an intersection point of the first adhesive strip and the second adhesive strip, an intersection point of the first adhesive strip and the third adhesive strip. For example, the first non-stick plastic film 3″ is overlapped with the second non-stick plastic film 4″ at the first corner end, and half of the overlapped portion of the first non-stick plastic film 3″ is tilted up and stick together with that of the second non-stick plastic film 4″ that is tilted up at the side facing the adhesive strip to form a first protrusion. Similarly, the first non-stick plastic film 3″ is overlapped with the third non-stick plastic film 20″ at the second corner end, and half of the overlapped portion of the first non-stick plastic film 3″ is tilted up and stick together with that of the third non-stick plastic film 20″ that is tilted up at the side facing the adhesive strip to form a second protrusion.

Due to the continuous non-stick plastic films design, all of the first non-stick plastic film 3″, the second non-stick plastic film 4″ and the third non-stick plastic film 20″ can be ripped off at one time by pulling the first protrusion or the second protrusion, thereby simplifying the operation and make it more convenient and more efficient.

In an implementation, the second protrusion and the first protrusion can be replaced with a non-stick plastic crease. In this case, at least one corner end of the continuous non-stick plastic film is formed with a non-stick plastic crease; where the at least one corner end of the continuous non-stick plastic film corresponds to at least one of: an intersection point of the first adhesive strip and the second adhesive strip, an intersection point of the first adhesive strip and the third adhesive strip, thereby further simplifying the operation and make it more convenient and more efficient.

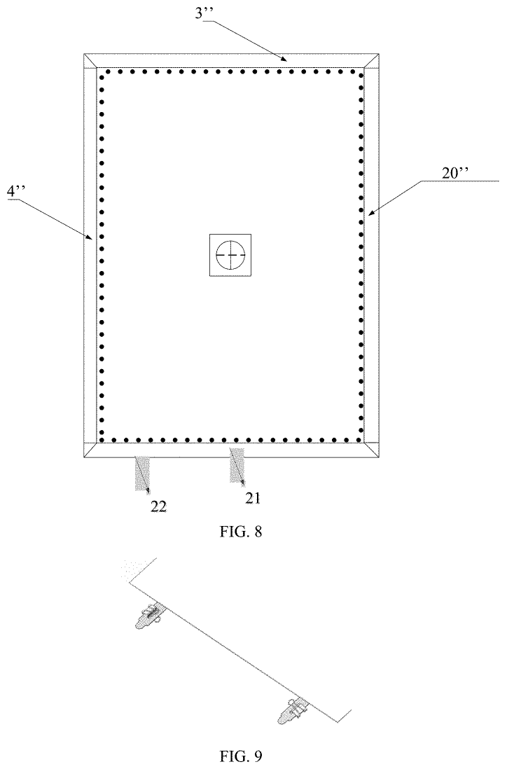

FIG. 8 is a schematic structural diagram of a colonoscopy auxiliary device with continuous non-stick plastic film according to another embodiment of the present disclosure. Based on the embodiments illustrated in FIG. 7, the colonoscopy auxiliary device further includes a fourth adhesive strip 22 and the fourth auxiliary adhesive strip (not shown), the continuous non-stick plastic film is configured to cover the first adhesive strip, the second adhesive strip, the third adhesive strip and the fourth adhesive strip. For example, a fourth non-stick plastic film 21 covering the fourth adhesive strip and the fourth auxiliary adhesive strip. Reference may be made to the above device embodiments for the operations of the related device and the similar expressions. As illustrated in FIG. 8, the fourth non-stick plastic film 21 is connected to the second non-stick plastic film 4″ at the third corner end, and the fourth non-stick plastic film 21 is connected to the third non-stick plastic film 20″ at the fourth corner end. In some embodiments, the fourth adhesive strip and the fourth auxiliary adhesive strip can instead the fourth tape 6 and the fourth tape 8 to help to close off the device.

At least one corner end of the continuous non-stick plastic film is formed with a protrusion, where the at least one corner end of the continuous non-stick plastic film corresponds to at least one of: an intersection point of the first adhesive strip and the second adhesive strip, an intersection point of the first adhesive strip and the third adhesive strip, an intersection point of the fourth adhesive strip and the second adhesive strip, and an intersection point of the fourth adhesive strip and the third adhesive strip. For example, the fourth non-stick plastic film 21 is overlapped with the second non-stick plastic film 4″ at the third corner end, and half of the overlapped portion of the fourth non-stick plastic film 21 is tilted up and stick together with that of the second non-stick plastic film 4″ that is tilted up at the side facing the adhesive strip to form a third protrusion. Similarly, the fourth non-stick plastic film 21 is overlapped with the third non-stick plastic film 20″ at the fourth corner end, and half of the overlapped portion of the fourth non-stick plastic film 21 is tilted up and stick together with that of the third non-stick plastic film 20″ that is tilted up at the side facing the adhesive strip to form a fourth protrusion.

Due to the continuous non-stick plastic films design, all of the first non-stick plastic film 3″, the second non-stick plastic film 4″, the third non-stick plastic film 20″ and the fourth non-stick plastic film 21 can be ripped off at one time by pulling any one of the first protrusion, the second protrusion, the third protrusion and the fourth protrusion, thereby simplifying the operation and make it more convenient and more efficient.

In an implementation, the second protrusion and the first protrusion are replaced with a non-stick plastic crease. In this case, at least one corner end of the continuous non-stick plastic film is formed with a non-stick plastic crease, where the at least one corner end of the continuous non-stick plastic film corresponds to at least one of: an intersection point of the first adhesive strip and the second adhesive strip, an intersection point of the first adhesive strip and the third adhesive strip, an intersection point of the fourth adhesive strip and the second adhesive strip, and an intersection point of the fourth adhesive strip and the third adhesive strip, thereby further simplifying the operation and make it more convenient and more efficient.

FIG. 9 is a schematic structural diagram of the fourth tape according to another embodiment of the present disclosure. At least one of the third tapes 5 and 7, and the fourth tapes 6 and 8 can be configured with a slender opening in the central portion, and a clip can get through the slender opening, and attach the corresponding tape(s) to a bed sheet, avoiding the device from coming off too easily during the operation.

Due to the continuous non-stick plastic films design, all of the first non-stick plastic film 3″, the second non-stick plastic film 4″, the third non-stick plastic film 20″ and the fourth non-stick plastic film 21 can be ripped off at the same time by pulling the first protrusion or the second protrusion, thereby further simplifying the operation and make it more convenient and more efficient.

Using the colonoscopy auxiliary device according to embodiments of the present disclosure, with the various structures descripted above, firstly, the GI physician's exposure to the stool and/or flatus can be significantly decreased; secondly, as the pandemic continues, the device will be a useful tool to protect doctors and/or staff; thirdly, if another viral or other type of illnesses arise in the future, it will be useful; fourthly, such device is inexpensive enough so that medical centers will not be burdened with cost, and the cost during COVID can be reduced; sixthly, even after initial surge of COVID dies down, health systems around the world will be very cautious for some time; seventhly, even after the pandemic, it can be used for routine procedures and colon decompression cases where there can be increased amount of stool exposure.

It should be noted that the above embodiments are merely intended for illustrating the technical solutions of the present application, but not for limiting thereto; although the present application is illustrated in detail with reference to the foregoing embodiments, those skilled in the art should understand that they may still make modifications to the technical solutions described in the foregoing embodiments or make equivalent substitutions to part or all technical features thereof, however, these modifications or substitutions do not make the essence of the corresponding technical solutions depart from the colonoscopy of the solutions of the embodiments of the present application.

What is claimed is:

1. A colonoscopy auxiliary device, comprising:
a foldable shield portion provided with a colo-examination aperture; and
a first adhesive strip disposed on a first edge of the foldable shield portion.

2. The colonoscopy auxiliary device according to claim 1, further comprising a second adhesive strip disposed on a second edge of the foldable shield portion, wherein the second adhesive strip intersects with the first adhesive strip, or an extension line of the second adhesive strip intersects with an extension line of the first adhesive strip.

3. The colonoscopy auxiliary device according to claim 2, wherein the foldable shield portion is a quadrilateral sheet, the first adhesive strip and the second adhesive strip are disposed on the first edge and the second edge of the foldable shield portion that are adjacent to each other, respectively,
wherein the quadrilateral sheet is a rectangle sheet or a square sheet.

4. The colonoscopy auxiliary device according to claim 3, further comprising at least one auxiliary adhesive strip, wherein the at least one auxiliary adhesive strip comprises at least one of a first auxiliary adhesive strip and a second auxiliary adhesive strip; wherein,
the first auxiliary adhesive strip is disposed on the first edge, wherein the first adhesive strip and the first auxiliary adhesive strip are disposed in parallel, and a first perforation line is disposed on the foldable shield portion and located between the first adhesive strip and the first auxiliary adhesive strip; and
the second auxiliary adhesive strip is disposed on the second edge, wherein the second adhesive strip and the second auxiliary adhesive strip are disposed in parallel, and a second perforation line is disposed on the foldable shield portion and located between the second adhesive strip and the second auxiliary adhesive strip.

5. The colonoscopy auxiliary device according to claim 1, wherein an area surrounding the colo-examination aperture is thicker than an other portion of the foldable shield portion;
wherein the area surrounding the colo-examination aperture has a multiple-layer structure, or has a single layer with thicker polymer.

6. The colonoscopy auxiliary device according to claim 1, further comprising a flap, wherein the flap is configured to cover the colo-examination aperture.

7. The colonoscopy auxiliary device according to claim 6, further comprising a handle element, wherein the flap is disposed on the foldable shield portion, and the handle element is disposed on an upper surface of the flap for moving the flap to cover or expose the colo-examination aperture.

8. The colonoscopy auxiliary device according to claim 6, wherein:
a first tape is disposed around the colo-examination aperture and configured to fix the flap over the colo-examination aperture to cover the colo-examination aperture; or
the flap is fixed rotatably on the foldable shield portion to cover and expose the colo-examination aperture alternatively.

9. The colonoscopy auxiliary device according to claim 1, wherein a third perforation line is disposed on the foldable shield portion from an edge of the colo-examination aperture to an edge of the foldable shield portion opposite to the first edge.

10. The colonoscopy auxiliary device according to claim 9, further comprising a second tape, wherein the second tape is disposed along the third perforation line and configured to reattach the third perforation line after the third perforation line is ripped.

11. The colonoscopy auxiliary device according to claim 1, further comprising a chuck portion, wherein the chuck portion is connected to an edge of the foldable shield portion opposite to the first edge.

12. The colonoscopy auxiliary device according to claim 11, wherein a fourth perforation line is disposed on a connection portion of the chuck portion and the foldable shield portion to separate the chuck portion and the foldable shield portion.

13. The colonoscopy auxiliary device according to claim 11, wherein the chuck portion comprises a back sheet and a top sheet, and the top sheet is attached on the back sheet;
wherein the back sheet is made of polypropylene, and the top sheet is made of hydrophilic non-woven.

14. The colonoscopy auxiliary device according to claim 12, further comprising:
a string disposed around a perimeter of the foldable shield portion and the chuck portion to string the foldable shield portion and the chuck portion together, and exposed from positions on a second edge of the foldable shield portion and an edge opposite to the second edge to facilitate the string being pulled,
wherein the positions correspond to the fourth perforation line.

15. The colonoscopy auxiliary device according to claim 1, wherein two third tapes are disposed on a second edge and an edge opposite to the second edge, respectively.

16. The colonoscopy auxiliary device according to claim 1, wherein at least one fourth tape is disposed on an edge of the foldable shield portion opposite to the first edge.

17. The colonoscopy auxiliary device according to claim 1, wherein the foldable shield portion comprises:
an absorbent layer and a water barrier layer, wherein the first adhesive strip is disposed on the absorbent layer; or
an absorbent layer, a water barrier layer and a non-woven layer, wherein the water barrier layer is disposed between the absorbent layer and the non-woven layer, and the first adhesive strip is disposed on the absorbent layer.

18. The colonoscopy auxiliary device according to claim 1, wherein the foldable shield portion comprises a colo-examination area, wherein the colo-examination aperture is disposed in the colo-examination area, and at least the colo-examination area of the foldable shield portion is transparent.

19. The colonoscopy auxiliary device according to claim 3, further comprising a third adhesive strip disposed on an edge of the foldable shield portion opposites to the second edge.

20. The colonoscopy auxiliary device according to claim 3, further comprising at least one of a third adhesive strip and a fourth adhesive strip, wherein:
the third adhesive strip is disposed on an edge of the foldable shield portion opposite to the second edge, and the device further comprises a continuous non-stick plastic film, wherein the continuous non-stick plastic film is configured to cover the first adhesive strip, the second adhesive strip, the third adhesive strip; and
the fourth adhesive strip is disposed on an edge of the foldable shield portion opposite to the first edge, and the device further comprises a continuous non-stick plastic film, wherein the continuous non-stick plastic film is configured to cover the first adhesive strip, the second adhesive strip, the third adhesive strip and the fourth adhesive strip.

21. The colonoscopy auxiliary device according to claim 20, wherein at least one corner end of the continuous non-stick plastic film is formed with a protrusion,
wherein the at least one corner end of the continuous non-stick plastic film corresponds to at least one of: an intersection point of the first adhesive strip and the second adhesive strip, an intersection point of the first adhesive strip and the third adhesive strip, an intersection point of the fourth adhesive strip and the second adhesive strip, and an intersection point of the fourth adhesive strip and the third adhesive strip.

22. The colonoscopy auxiliary device according to claim 20, wherein at least one corner end of the continuous non-stick plastic film is formed with a non-stick plastic crease;
wherein the at least one corner end of the continuous non-stick plastic film corresponds to at least one of: an intersection point of the first adhesive strip and the second adhesive strip, an intersection point of the first adhesive strip and the third adhesive strip, an intersection point of the fourth adhesive strip and the second adhesive strip, and an intersection point of the fourth adhesive strip and the third adhesive strip.
